(19) United States
(12) Patent Application Publication
(10) Pub. No.: US 2008/0161702 A1
(43) Pub. Date: Jul. 3, 2008

(54) EXAMINING DEVICE FOR EXAMINING AN AORTIC VALVE AND A PULMONARY VALVE COMPETENCY

(76) Inventors: Jen-Ping CHANG, Niaosong Hsiang (TW); Chi-Nan TSENG, Tainan City (TW); Yuh-Der LIAO, Kaohsiung (TW)

Correspondence Address: PATENTTM.US, P. O. BOX 82788, PORTLAND, OR, 97282-0788, US

(21) Appl. No.: 11/619,032

(22) Filed: Jan. 2, 2007

(51) Int. Cl. A61B 5/00

(52) U.S. Cl. 600/508

(57) ABSTRACT

An examining device for an aortic valve and a pulmonary valve competency has a cylinder and a liquid infusion catheter assembly. The cylinder has a proximal end, a distal end, a liquid channel and an air channel. The proximal end has a first opening and a transparent lens on the proximal end and sealing the first opening. The distal end has a second opening communicating with the first opening. The liquid channel has a liquid intake hole and a liquid outtake hole communicating with the liquid intake hole. The air channel has a first air intake hole and an air outtake hole communicating with the air intake hole. The liquid infusion catheter assembly connects with the proximal end of the cylinder. The examining device allows surgeons to immediately examine whether a surgery for valves is successful.

### BACKGROUND OF THE INVENTION

1. Field of Invention

The present invention relates to an examining device for examining an aortic valve and a pulmonary valve competency during a reparative valve surgery.

2. Description of the Related Art

A human heart has four chambers and four valves. The chambers include a right atrium, a right ventricle, a left atrium and a left ventricle. The valves are a tricuspid valve, a pulmonary valve, a mitral valve and an aortic valve. The tricuspid valve is located between the right atrium and the right ventricle. The pulmonary valve is located between the right ventricle and the main pulmonary artery. The mitral valve is located between the left atrium and the left ventricle. The aortic valve is located between the left ventricle and the aorta. The great vessel means aorta or main pulmonary artery. As blood leaves each chamber, the blood passes through a valve. If the valve is normal and is competent, the valve will make sure the blood flows only in a forward direction. However, if the valve is abnormal and is incompetent, the blood will flow backwards (regurgitation) and cause heart murmur, angina pectoris, dyspnea, cardiomegaly, heart failure or even death.

When a patient's aortic valve or pulmonary valve is diseased, the aortic valve or the pulmonary valve can be replaced by a prosthetic valve or the patient can take a reparative procedures to repair the valve. However, results of reparative procedures are superior to results of replacement. An open heart operation is performed under extracorporeal circulation and cardioplegic cardiac arrest. In general, a valve competency after a reparative surgery is unable to be confirmed with echocardiography during the cardioplegic cardiac arrest, unless the heart is reperfused and heartbeats are restored. Therefore, if the valvular incompetency is detected after the operation, a second operation under repeated cardioplegic cardiac arrest is mandatory and this carries significant risks.

To overcome the shortcomings, the present invention provides an examining device for examining an aortic valve and a pulmonary valve competency during cardioplegic cardiac arrest to mitigate or obviate the aforementioned.

### SUMMARY OF THE INVENTION

The primary objective of the present invention is to provide an examining device for examining an aortic valve and a pulmonary valve competency during cardioplegic cardiac arrest for a reparative valvular surgery.

To achieve the objective, the examining device for examining an aortic valve and a pulmonary valve contents a cylindrical structure with 2 build-in channels and a liquid infusion catheter assembly, wherein one of the 2 build-in channels is for liquid infusion and another is for air venting. The cylindrical structure has a proximal end, a distal end, a liquid channel and an air channel. The proximal end has a first opening and a transparent lens. The first opening of the cylindrical structure is defined through the proximal end of the cylindrical structure. The transparent lens is mounted on the proximal end and seals the first opening. A distal end has a second opening. The second opening is defined through the distal end and communicates with the first opening. The liquid channel has a liquid intake hole and a liquid outtake hole. The liquid intake hole is formed in the proximal end. The liquid outtake hole is formed in the distal end and communicates with the liquid intake hole. The air channel has a first air intake hole and an air outtake hole. The first air intake hole is defined through the distal end. The air outtake hole is defined through the proximal end and communicates with the air intake hole. The liquid infusion catheter assembly connects with the proximal end of the cylinder. After finishing the reparative valvular procedure, surgeons can examine whether the surgery is success or not by using the examining device during the cardiac arrest. If the procedure is failed, surgeon can deal with problems at once, so operative risks can be minimized.

Other objectives, advantages and novel features of the invention will become more apparent from the following detailed description when taken in conjunction with the accompanying drawings.

### BRIEF DESCRIPTION OF THE DRAWINGS

FIG. 1 is an operational view in partial section of an examining device for examining an aortic valve and a pulmonary valve in accordance with the present invention mounted in a great vessel.

FIG. 2 is an enlarged cross sectional side view of the examining device for examining the aortic valve and the pulmonary valve in FIG. 1 without a liquid infusion catheter assembly and an air venting catheter assembly;

FIG. 3 is a perspective view of the examining device for examining the aortic valve and the pulmonary valve in FIG. 2 without an autoinflatable balloon;

FIG. 4 is a perspective view of the examining device for examining the aortic valve and the pulmonary valve in FIG. 2; and

FIG. 5 is an operational side view in partial section of the examining device for examining the aortic valve and the pulmonary valve in FIG. 1 after liquid flows into the space through a liquid infusion catheter assembly; and

FIG. 6 is an operational view in partial section of the examining device for examining the aortic valve and the pulmonary valve in FIG. 1 mounted in the great vessel and out of the aortic valve or the pulmonary valve.

### DETAILED DESCRIPTION OF THE INVENTION

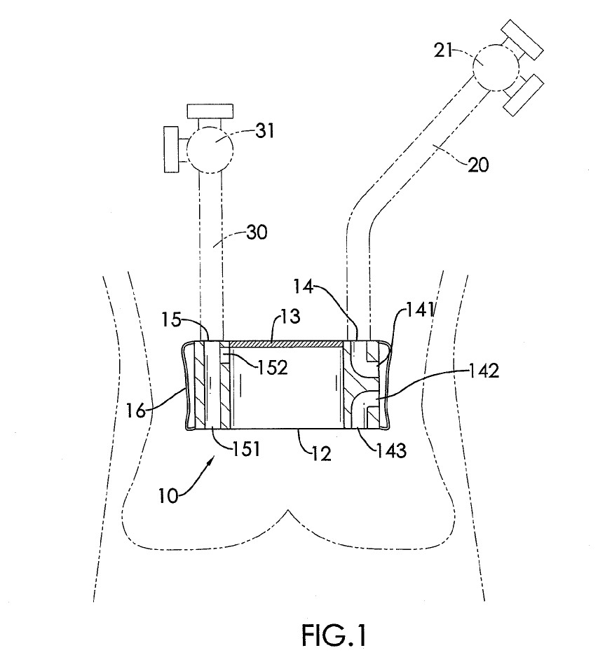

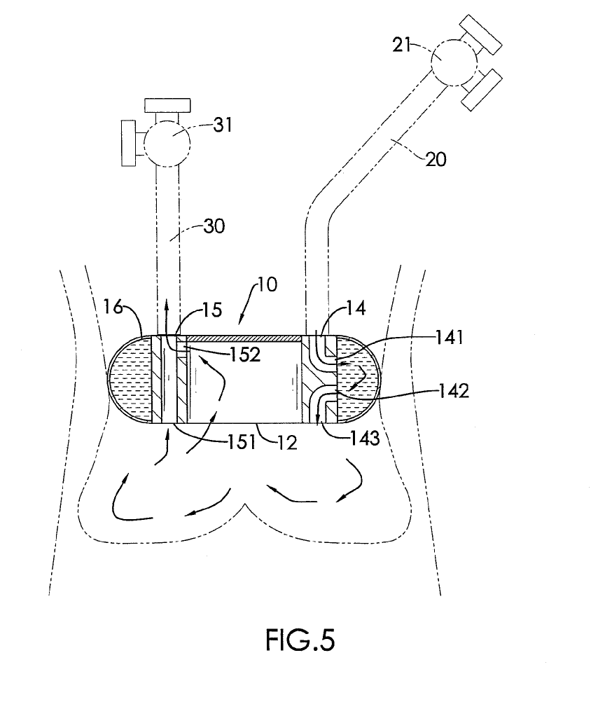

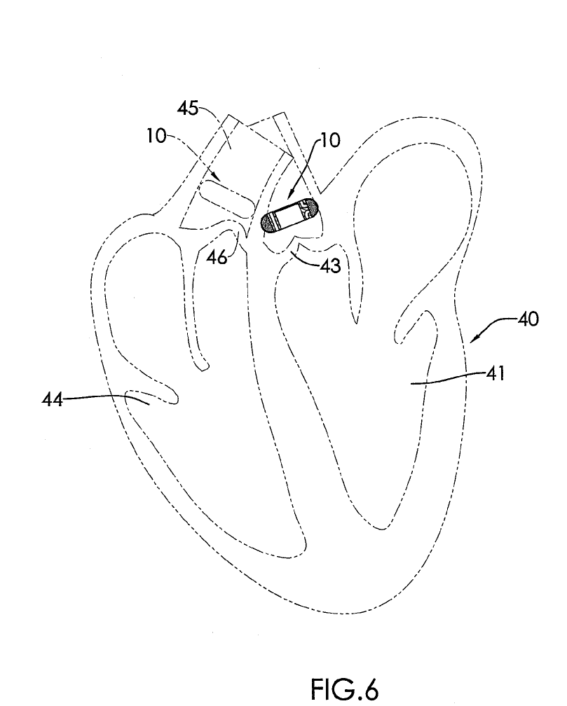

With reference to FIGS. 1, 5 and 6, an examining device for examining an aortic valve (43) and a pulmonary valve (46) in accordance with the present invention is mounted in the aortic (42) or the pulmonary root (45) and above the valve (43, 46). A space is formed between the valve (43, 46) and the examining device. The examining device has a hollow cylinder (10), a liquid infusion catheter assembly and an air venting catheter assembly.

The cylinder (10) has a central hole, a proximal end, a distal end, an outer surface, an inner surface, a liquid channel and an air channel.

The central hole is defined axially through the cylinder (10).

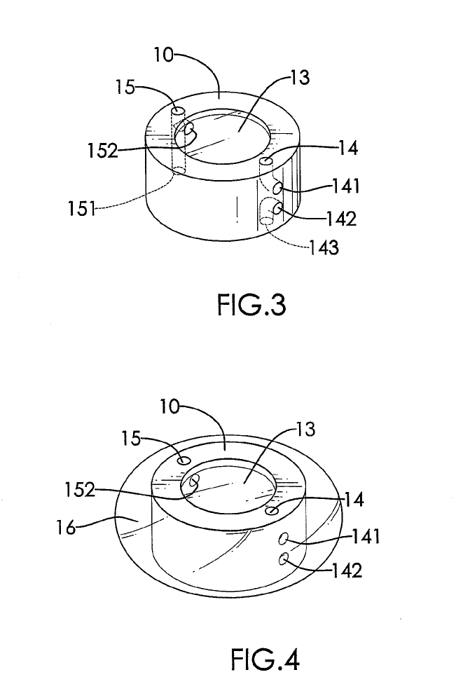

With further reference to FIG. 3, the proximal end has a first opening, a transparent lens (13), a liquid intake hole (14) and an air outtake hole (15). The first opening is defined through proximal end of the cylinder (10) and communicates with the central hole. The transparent lens (13) is mounted on the proximal end and seals the first opening.

The distal end has a second opening (12), a liquid outtake hole (143) and a first air intake hole (151). The second opening (12) is defined through the distal end and communicates with the first opening of the proximal end and the central hole.

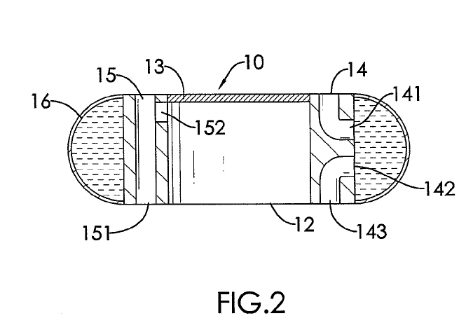

With further reference to FIGS. 2 and 4, the outer surface has an autoinflatable balloon (16). The autoinflatable balloon (16) is made of silica gel, is mounted on the outer surface and covers the outer surface.

The liquid channel is defined through the cylinder (10) and comprises a liquid intake hole (14) and a liquid outtake hole (143). The liquid intake hole (14) is formed in the proximal end of the cylinder (10) and allow liquid to flow through the cylinder (10) into the space. The liquid outtake hole (143) is formed in the distal end of the cylinder (10) and communicates with the liquid intake hole (14). The liquid channel may have a first bypass and a second bypass to allow liquid flow into and expand the autoinflatable balloon (16). The first bypass has an inside liquid outtake hole (141). The inside liquid outtake hole (141) is formed in the outer surface, communicates with the liquid intake hole (14) and allows liquid to flow in the autoinflatable balloon (16) from the liquid intake hole (14) and the inside liquid outtake hole (141). Thus, when the autoinflatable balloon (16) filled with liquid, the autoinflatable balloon (16) will expand and abut a great vessels including the aortic (42) and the pulmonary root (45), so the examining device can be mounted in the great vessels (42, 45) securely. The second bypass has an inside liquid intake hole (142). The inside liquid intake hole (142) is formed in the outer surface, communicates with the liquid outtake hole (143) and the autoinflatable balloon (16) and allows liquid in the autoinflatable balloon (16) to flow into the space between the examining device and the valve (43, 46) through the inside liquid intake hole (142) and the liquid outtake hole (143). Thus, when the space is filled with liquid, surgeons can observe a morphology of the valve leaflets from the transparent lens (13) whether the liquid is leaking or not. If the liquid in the space is leaking, the valve (43, 46) is incompetent. If the liquid in the space is not leaking, the valve (43, 46) is competent.

The air channel is defined through the cylinder (10) and has a first air intake hole (151), an air outtake hole (15) and a second air outtake hole (152). The first air intake hole (151) is defined through the distal end. The air outtake hole (15) is defined through the proximal end of the cylinder (10), communicates with the air intake hole (151) and allows air between the valve (43, 46) and the examining device to flow from the first air intake hole (151) to the air outtake hole (15). The inner surface has a second air outtake hole (152). The second air outtake hole (152) is formed in the inner surface of the cylinder (10) adjacent to the proximal end of the cylinder (10) and communicates with the air outtake hole (15). Air in the space can be released completely through the second air outtake hole (152). The liquid infusion catheter assembly connects with the proximal end of the cylinder (10) and has a catheter (20) and a 3-way occluder (21). The catheter (20) connects to and communicates with the liquid intake hole (14) and allows liquid to flow in the cylinder (10) from the catheter (20). The 3-way occluder (21) is mounted on the catheter (20) and controls a flow rate of the liquid in the catheter (20).

The air venting catheter assembly connects with the proximal end of the cylinder (10) and has a rigid catheter (30) and a 3-way occluder (31). The catheter (30) connects to and communicates with the air outtake hole (15), allows air to release out of the cylinder (10) through the catheter (30) and allows a surgeon to hold the examining device. The 3-way occluder (31) is mounted on the catheter (30) and controls a flow rate of the air in the catheter (30).

After completing the procedures for repairing a patient's aortic or pulmonary valve and before reperfusion, surgeons can examine whether the surgery is success or not by using the examining device during the cardioplegic cardiac arrest. If the repair is failed, surgeon can deal with problems at once, so the patient can avoid the risk of the repeated cardioplegic cardiac arrest.

Even though numerous characteristics and advantages of the present invention have been set forth in the foregoing description, together with details of the structure and function of the invention, the disclosure is illustrative only. Changes may be made in detail, especially in matters of shape, size and arrangement of parts within the principles of the invention to the full extent indicated by the broad general meaning of the terms in which the appended claims are expressed.

What is claimed is:

1. An examining device for examining an aortic valve and a pulmonary valve, and the examining device having
a cylinder having
a central hole defined axially through the cylinder;
a proximal end having
a first opening defined through proximal end of the cylinder and communicating with the central hole; and
a transparent lens mounted on the proximal end and sealing the first opening;
a distal end having
a second opening defined through the distal end and communicating with the first opening of the proximal end and the central hole;
a liquid channel defined through the cylinder and comprising
a liquid intake hole formed in the proximal end of the cylinder; and
a liquid outtake hole formed in the distal end of the cylinder and communicating with the liquid intake hole; and
an air channel defined through the cylinder and comprising
a first air intake hole defined through the distal end; and
an air outtake hole defined through the proximal end of the cylinder and communicating with the air intake hole; and
a liquid infusion catheter assembly connecting with the proximal end of the cylinder.

2. The examining device for examining an aortic valve and a pulmonary valve as claimed in claim 1, wherein
the cylinder further has
an outer surface comprising an autoinflatable balloon made of silica gel, mounted on the outer surface and covering the outer surface; and
the liquid channel comprises
an inside liquid outtake hole formed in the outer surface and communicating with the liquid intake hole; and
an inside liquid intake hole formed in the outer surface, communicating with the liquid outtake hole and the autoinflatable balloon.

3. The examining device for examining an aortic valve and a pulmonary valve as claimed in claim 1, wherein
the cylinder further has an inner surface; and
the air channel of the cylinder further having a second air outtake hole formed in the inner surface of the cylinder adjacent to the proximal end of the cylinder and communicating with the air outtake hole.

4. The examining device for examining an aortic valve and a pulmonary valve as claimed in claim 1, wherein the liquid infusion catheter assembly has
a catheter connecting to and communicating with the liquid intake hole; and
a 3-way occluder mounted on the catheter and adapted to control a flow rate of liquid in the catheter.

5. The examining device for examining an aortic valve and a pulmonary valve as claimed in claim 1 further having an air venting catheter assembly connecting with the proximal end of the cylinder and having
a rigid catheter connecting to and communicating with the air outtake hole; and
a 3-way occluder mounted on the rigid catheter and adapted to control a flow rate of air in the catheter.

6. The examining device for examining an aortic valve and a pulmonary valve as claimed in claim 2, wherein the cylinder further has an inner surface having a second air outtake hole formed in the inner surface of the cylinder adjacent to the proximal end of the cylinder and communicating with the air outtake hole.

7. The examining device for examining an aortic valve and a pulmonary valve as claimed in claim 2, wherein the liquid infusion catheter assembly has
a catheter connecting to and communicating with the liquid intake hole; and
a 3-way occluder mounted on the catheter and adapted to control a flow rate of liquid in the catheter.

8. The examining device for examining an aortic valve and a pulmonary valve as claimed in claim 2 further having an air venting catheter assembly connecting with the proximal end of the cylinder and having
a rigid catheter connecting to and communicating with the air outtake hole; and
a 3-way occluder mounted on the rigid catheter and adapted to control a flow rate of air in the catheter.
